(19) United States
(12) Patent Application Publication
(10) Pub. No.: US 2007/0149805 A1
(43) Pub. Date: Jun. 28, 2007

(54) Method for the extraction and purification of shikimic acid

(75) Inventors: Shiyou Li, Nacogdoches, TX (US); Wei Yuan, Nacogdoches, TX (US); Ping Wang, Nacogdoches, TX (US); Zhizhen Zhang, Nacogdoches, TX (US); Wanli Zhang, Nacogdoches, TX (US); Stacy Ownby, Nacogdoches, TX (US)

Correspondence Address: FULWIDER, PATTON, LEE & UTECHT, LLP;10877 WILSHIRE BLVD., LOS ANGELES,, LONG BEACH, CA, 900245615, US

(73) Assignee: STEPHEN F. AUSTIN STATE UNIVERSITY

(21) Appl. No.: 11/317,902

(22) Filed: Dec. 23, 2005

(51) Int. Cl. C07C 62/04

(52) U.S. Cl. 562/508

(57) ABSTRACT

Improved methods for producing shikimic acid and the use of sweetgum plant tissues in the production of shikimic acid.

### BACKGROUND OF THE INVENTION

1. Field of the Invention

Applicant's invention relates to production of pharmaceutical constituents from plant tissues.

2. Background Information

a. The Flu Threat.

Each year, the Centers for Disease Control and Prevention (CDC) estimate that between 5 percent and 20 percent of Americans will develop the flu. According to the CDC, about 200,000 people will be hospitalized due to flu complications, and as many as 36,000 will die as a result of the illness. Those most at risk from influenza are the elderly, children, and people with chronic health conditions.

Of current, far greater and more specific concern is the avian influenza, or “bird flu,” which is a contagious disease caused by avian (bird) influenza (flu) viruses that normally infect birds (and, less commonly, pigs). These influenza viruses occur naturally among birds. Wild birds worldwide carry the viruses in their intestines, but usually do not get sick from them. However, avian influenza is very contagious among birds and can make some domesticated birds, including chickens, ducks, and turkeys, very sick and kill them.

Unlike normal seasonal influenza, where infection causes only mild respiratory symptoms in most people, the disease caused by H5N1, in humans, follows an unusually aggressive clinical course, with rapid deterioration and a high mortality rate. In the present outbreak, more than half of those infected with the virus have died.

There are many different subtypes of type A influenza viruses. These subtypes differ because of changes in certain proteins on the surface of the influenza A virus (hemagglutinin [HA] and neuraminidase [NA] proteins). There are 16 known HA subtypes and 9 known NA subtypes of influenza A viruses. Many different combinations of HA and NA proteins are possible. Each combination represents a different subtype. All known subtypes of influenza A viruses can be found in birds.

During an outbreak of avian influenza among poultry, there is a proven risk to humans who have contact with infected birds or surfaces that have been contaminated with secretions or excretions from infected birds.

Influenza A (H5N1) virus—also called “H5N1 virus”—is an influenza A virus subtype that occurs mainly in birds, is highly contagious among birds, and can be deadly to them. Outbreaks of avian influenza H5N1 occurred among poultry in eight countries in Asia (Cambodia, China, Indonesia, Japan, Laos, South Korea, Thailand, and Vietnam) during late 2003 and early 2004. At that time, more than 100 million birds in the affected countries either died from the disease or were killed in order to try to control the outbreaks.

By March 2004, the H5N1 outbreak was reported to be under control. Since late June 2004, however, new outbreaks of H5N1 influenza among poultry were reported by several countries in Asia (Cambodia, China, Indonesia, Kazakhstan, Malaysia, Mongolia, Russia [Siberia], Thailand, and Vietnam). Influenza H5N1 infection also has been reported among poultry in Turkey and Romania and among wild migratory birds in Croatia. The current outbreaks of highly pathogenic avian influenza are the largest and most severe on record. Never before in the history of this disease have so many countries been simultaneously affected, resulting in the loss of so many birds.

Human cases of influenza A (H5N1) infection have been reported in Cambodia, China, Indonesia, Thailand, and Vietnam. More than 130 human cases have been reported by the World Health Organization since January 2004, and 70 people have so far died as a result of bird flu infection. Experts from around the world are watching the H5N1 situation in Asia and Europe very closely and are preparing for the possibility that the virus may begin to spread more easily and widely from person to person.

The widespread persistence of H5N1 in poultry populations poses two primary infection modalities for humans. The first, a present hazard, is that of direct infection when the virus passes from poultry to humans. A second anticipated modality of even greater concern (though not yet a reality) relates to an expected mutation of the H5N1 virus to a form that is highly infectious in a human-to-human mode, easily spreading from person to person (including by air). Such a mutation is expected to mark the onset of a global avian flu outbreak—a pandemic.

The H5N1 virus that has caused human illness and death in Asia is resistant to amantadine and rimantadine, two antiviral medications commonly used for influenza. On Dec. 16, 2005, the Ministry of Health in China confirmed an additional case of human infection with the H5N1 avian influenza virus. This is China's sixth laboratory-confirmed human case. Of these cases, two have been fatal. Currently, there is no commercially available vaccine to protect humans against H5N1 virus that is being seen in Asia and Europe. However, vaccine development efforts are taking place.

On Oct. 24, 2005, the Food and Drug Administration has announced formation of a “rapid response team” to ensure that antiviral drugs (discussed below) are available in the event there is a pandemic outbreak of avian flu.

b. Remedial Measures for Flu.

Vaccines are produced each year for seasonal influenza but will not protect against pandemic influenza.

Oseltamivir phosphate (one of only four available anti-influenza drugs) was the first orally active, commercially developed neuraminidase inhibitor. It was developed by Gilead Sciences and is currently marketed by Hoffman-La Roche (Roche) under the trademark TAMIFLU. Oseltamivir is a neuraminidase inhibitor used in the treatment and prophylaxis of both influenza A and influenza B, and is widely considered the most useful treatment against avian flu.

As an inhibitor of neuraminidase, which is essential for influenza virus replication, TAMIFLU is potent against the H5N1 and H7N7 virus strains. (TAMIFLU was widely used during the H5N1 avian influenza epidemic in Southeast Asia in 2005.) The major bottleneck in TAMIFLU production is the availability of shikimic acid, a naturally occurring chemical compound derived from plants, described below, which cannot be commercially synthesized.

C. Shortages of Effective Medications.

Supply shortages of neuraminidase inhibitors will represent the primary hurdle in their effective use in combating any pandemic, and presently, significantly impedes the world community's efforts to prepare for such a contingency. Such shortages arise primarily from limited production capacity, but also from cost factors, particularly in the case of economically challenged countries.

At present, manufacturing capacity (which has recently quadrupled) can only supply oseltamivir to treat 20% of the world's population in ten years' time. The United States currently cannot even ensure TAMIFLU for 1% of its population, although many claim the U.S. should have it available for at least 10% of the population. “As yet we have not identified anyone who could significantly speed up the agreed delivery timelines (of TAMIFLU) for the first half of 2006 . . . ” Roche Pharma CEO William Burns said on Dec. 12, 2005.

Roche also said it had signed the first sub-licensing agreement for the overall production of oseltamivir for pandemic use, with the Shanghai Pharmaceutical Group in China, and is in further negotiations for local partnerships in other countries.

Under even the best of circumstances, the manufacturing process for oseltamivir is complex and time-consuming, and is not easily transferred to other facilities.

Specifically, oseltamivir synthesis commences from naturally available shikimic acid. The 3,4-pentylidene acetal mesylate is prepared, according to one method in three steps: (1) esterification with ethanol and thionyl chloride; (2) ketalization with para-toluenesulfonic acid and 3-pentanone; and (3) methylation with triethylamine and methanesulfonyl chloride. This synthesis avoids the use of potentially explosive azide reagents and intermediates; however, the synthesis actually used by Roche uses azides. Roche has other routes to TAMIFLU that do not involve the use of shikimic acid as a chiral pool starting material, such as a Diels-Alder route involving furan and ethyl acrylate, or a isophthalic acid route which involves catalytic hydrogenation and enzymatic desymmetrization.

d. The Root of the Problem.

In early 2005, Roche announced a TAMIFLU production shortage. According to Roche, the primary disruptive factor in oseltamivir production is an inadequate supply of shikimic acid—a substance which cannot now be economically synthesized, and is only effectively isolated from the fruit of the Chinese star anise (Illicium verum). A shortage of star anise is one of the key factors behind the worldwide shortage of TAMIFLU (as of 2005).

When extracted from star anise, shikimic acid is extracted from the seeds in a ten-stage manufacturing process. Approximately 90% of the harvest is already used by Roche in making TAMIFLU.

Although many autotrophic organisms produce shikimic acid, the isolation yield is low when effected by any presently known process and involving plants thus far known to have been evaluated as a source for shikimic acid.

e. Strategic and Economic Threats from a Lack of Viable Alternatives to Conventional, Remedial Measures.

Of strategic and economical significance for the United States, as well as most of the world's nations, star anise is grown almost exclusively in four provinces in China, and only harvested between March and May. Compounding this problem is that the trees do not begin producing fruits until they are eight to ten years of age. Of ominous significance, China has recognized the strategic and economic importance of star anise, and has taken steps to secure its resources.

f. To-Date, Inadequate Steps to Provide Alternative Sources of Shikimic Acid.

Because of the dire need for alternative sources of shikimic acid, researchers have attempted to isolate it from a number of other plant species, using a variety of methods. Some alternative methods have surfaced, but fall well short of the present invention (to be described below) with respect to yield, cost considerations, and/or realistic sources of required substrate(s).

One alternative method for production of shikimic acid involves the relatively expensive and time-consuming process of fermentation of genetically modified bacteria. (e.g., U.S. Pat. No. 6,613,552 issued to Frost, et al. on Sep. 2, 2003 entitled “Biocatalytic synthesis of shikimic acid”) Frost, et. al. describe a recombinant microbe-catalyzed conversion of a carbon source to acid. On the other hand, U.S. Pat. No. 6,436,664 issued to lomantas, et al. on Aug. 20, 2002, entitled “Method for producing shikimic acid”, provides a method for producing shikimic acid by direct fermentation and a microorganism that is used in the method.

These alternative approaches to providing shikimic acid are woefully inadequate to address the present shortages, much less the anticipated, future pandemic-related shortages.

g. An Objective of Compelling Significance.

In light of the above, urgent need exists for a practicable, alternative methods for producing large quantities of shikimic acid from sources other than star anise. Even more beneficial would be such a method which is cost-effective and/or merely requires supplies of plentiful starting materials. Add to such characteristics environmentally benign use and byproducts, and the societal benefits of such an method would rival the most heralded of inventions.

h. An Introduction to Sweetgum (Liquidambar styraciflua).

Sweetgum (Liquidambar styraciflua) is one of the most important hardwoods in the southeastern United States (Harris 2003). Its growing range extends from Connecticut southward throughout the east to central Florida and eastern Texas. It may be found as far west as Missouri, Arkansas, and Oklahoma to as far north as southern Illinois. It also grows in scattered locations in northeastern and central Mexico, Guatemala, Belize, El Salvador, Honduras, and Nicaragua (Duncan and Duncan 1987; Kormanik 1990). It is also cultivated in Hawaii (St. John 1973).

Sweetgum is often the pioneer hardwood species to move into an abandoned field or logged-out area and has a tendency to quickly spread. Sweetgum is generally free from pests and diseases and is drought resistant. It is tolerant of different soil types but grows best on rich, moist, alluvial clay and loamy soils associated with river bottoms (Lea and Frederick 1990). Following logging or prescribed burn events, sweetgum will sprout from the stump or root crown. Although seedlings reach a height of 1.5 m in three to five years, sprouts often reach this height in one growing season. Sweetgum fruits ripen from September through November and persist through the winter (Kormank 1990).

Sweetgum has a long history of being utilized for medicinal purposes. It produces a balsamic oleo-resin called “storax,” which can be chewed as a gum. Medicinally the gum has been used for catarrh, coughs, dysentery, sores, and treatment of wounds. Native Americans used it as an anti-diarrheal, dermatological aid, and sedative (Moerman 1986). The balsam, collected from the inner bark, is currently used in soaps and cosmetics, as a fixative in perfumes, adhesives, lacquers, and incense, and as a flavoring in tobacco. Sweetgum wood is often used in cabinet and furniture making.

Despite all of the above, sweetgum is not known to have been recognized, through employing any method or process, as a practicable source of shikimic acid.

### SUMMARY OF THE INVENTION

In view of the foregoing, it is an object of the present invention to provide a method for the production of shikimic acid.

It is another object of the present invention to provide a new source for shikimic acid.

It is another object of the present invention to provide a method to successfully isolate or extract shikimic acid from specific tissues.

It is another object of the present invention to provide a method to purify shikimic acid isolated from specific tissues.

It is another object of the present invention to provide a method for the production of shikimic acid that is simple and fast.

It is another object of the present invention to provide a rapid method for the analysis of shikimic acid.

It is another object of the present invention to provide a method for the production of shikimic acid that is far most cost-effective than conventional processes for producing same.

It is another object of the present invention to provide a simple, fast and cost-effective method for the production of shikimic acid that is readily adaptable for commercial application.

It is another object of the present invention to provide a method for producing shikimic acid in a more environmentally benign manner than presently-available methods of shikimic acid production.

In satisfaction of these and related objectives, the present inventors have invented processes for the application of certain extraction methods to species of Sweetgum (Liquidambar styraciflua L.) and close relatives thereof to produce shikimic acid. The processes of the present invention (which are preferentially applied to leaves, fruits, and intact clippings [annual stems with leaves]), produce shikimic acid at previously unattainable yield levels, from previously untapped, yet plentiful substrates. Remarkably, this new pathway to rectifying life-threatening supply shortages of shikimic acid is actually more cost effective than any presently known method for producing same.

A particularly beneficial purification method (following extraction) yields the optimal, over-all shikimic acid production according to the preferred mode of the present invention. Therefore, the combination of substrate choice, extraction method and purification methods, all as taught herein, provides a remarkable advance over any known art involving the production of shikimic acid, or any intermediary or interim step involved therewith.

Even if the yield levels and cost considerations of the present invention were not as favorable as they are, the ability to use Sweetgum (and its close relatives) as a practical alternative source of shikimic acid (at any yield or price point) would still provide advantages which may be characterized as quite literally bearing on national security. If the dreaded avian flu pandemic becomes reality, the United States' access to currently supplies of shikimic acid will almost certainly be curtailed, if not altogether eliminated.

The processes of the present invention are highly beneficial, even with access to present sources of shikimic acid. While 30 Kg of star anise fruit (the current global supply source of shikimic acid) produce 3 Kg of shikimic acid, the supply of such fruit is very limited. Furthermore, the present inventors have demonstrated that the leaves and stems of Illicium (still not a plentiful resource) contain less than 1% shikimic acid. In stark contrast, the processes of the present invention, utilizing very plentiful sweetgum leaves, fruits, and stems, provide over-all yields up to 6.15%.

The methods of the present invention, because of its reliance, as the primary reagent, only upon the domestically abundant Sweetgum, its high yield, and cost-effectiveness will readily generate enough shikimic acid to facilitate the production of anticipated global requirements for TAMIFLU. The present inventors conservatively estimate that current natural resources in East Texas alone could produce a sufficient quantity of shikimic acid to meet the TAMIFLU demand for the entire U.S. population.

### BRIEF DESCRIPTION OF THE DRAWINGS

FIG. 1 is a table illustrating the gradient condition of the mobile phase of HPLC analysis. The buffer solution was 0.01M K₂HPO₄, pH 2.5 adjusted by phosphoric acid.

FIG. 2 is a table illustrating the intra- and inter-day accuracy and precision of shikimic acid standard.

FIG. 3 is a table illustrating the mean (±s.e.) shikimic acid recoveries from fruit hulls following three extraction methods (mesic habitat, Nacogdoches, Tex., USA) (solvent: methanol) (%, dry wt).

FIG. 4 is a table illustrating the mean (±s.e.) shikimic acid recoveries from fruit hulls extracted on ASE 200 with different solvents (mesic habitat, Nacogdoches, Tex., USA) (%, dry wt).

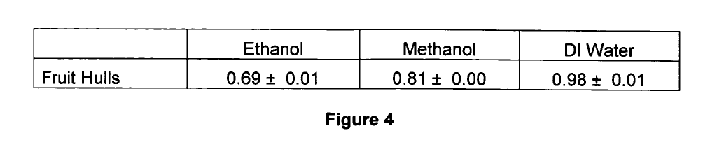

FIG. 5 is a table illustrating the mean (±s.e.) shikimic acid recoveries from yellow leaves following two extraction methods (wet habitat, Nacogdoches, Tex., USA) (solvent: DI water) (%, dry wt).

FIG. 6 is a table illustrating the mean (±s.e.) shikimic acid recoveries from tissues of different species and varieties of Liquidambar (wet habitat, Nacogdoches, Tex., USA) (solvent: DI water) (%, dry wt).

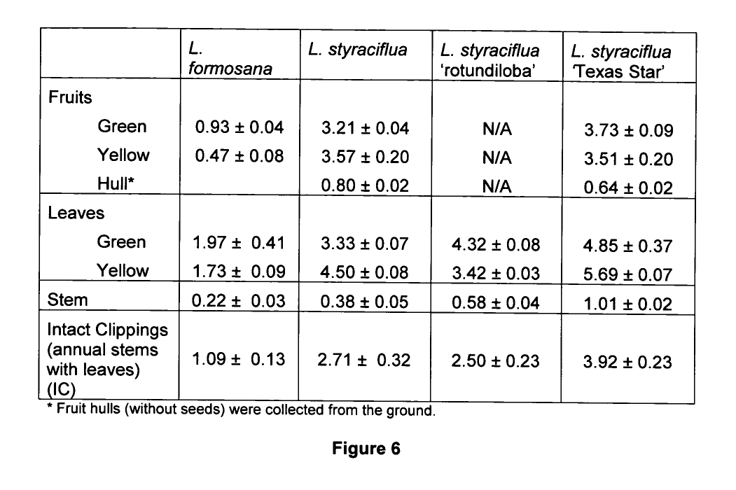

FIG. 7 is a table illustrating the mean (±s.e.) shikimic acid recoveries from L. styraciflua trees from different habitats (Nacogdoches, Tex., USA) (solvent: DI water) (%, dry wt).

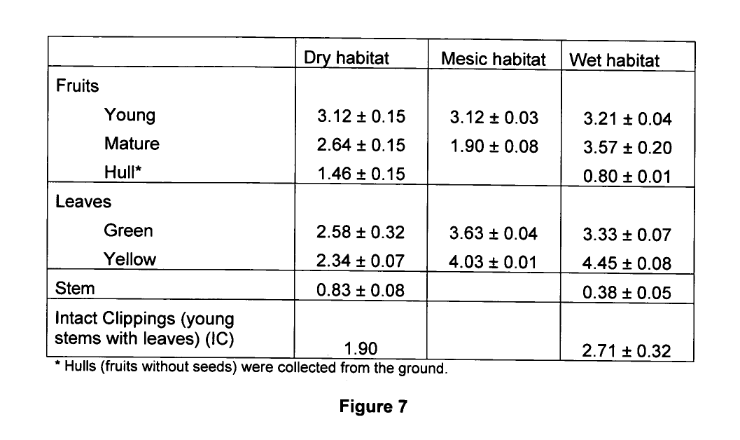

FIG. 8 is a table illustrating the mean (±s.e.) shikimic acid recoveries from L. styraciflua tissues following different extraction methods (%, dry wt).

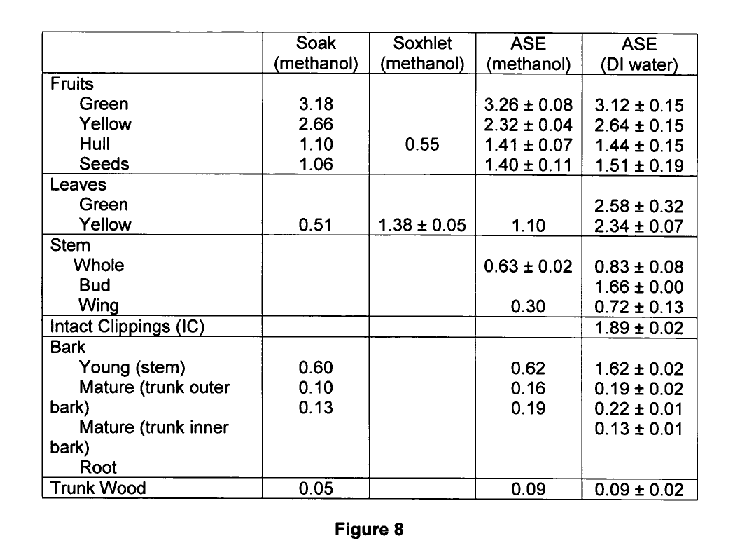

FIG. 9 is a table illustrating the mean (±s.e.) shikimic acid recoveries from different aged leaves of L. styraciflua (Nacogdoches, Tex., USA) (solvent: DI water) (%, dry wt).

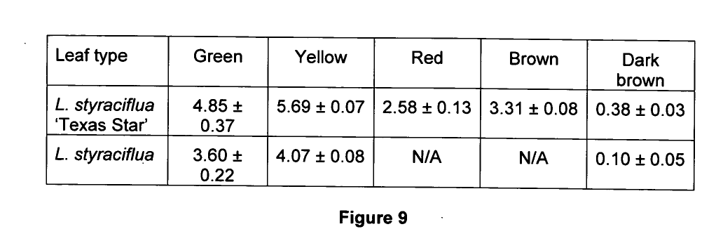

FIG. 10 is a table illustrating the mean (±s.e.) shikimic acid recoveries from leaves of parent tree and coppice offspring (mesic habitat, Nacogdoches, Tex., USA) (solvent: DI water) (%, dry wt).

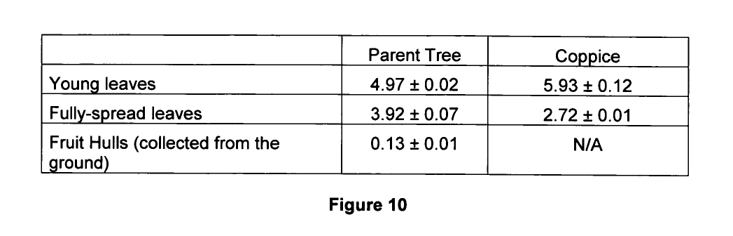

FIG. 11 is a table illustrating the mean (±s.e.) shikimic acid recoveries from coppice trees following a prescribed burn (mesic habitat, Nacogdoches, Tex., USA) (solvent: DI water) (%, dry wt).

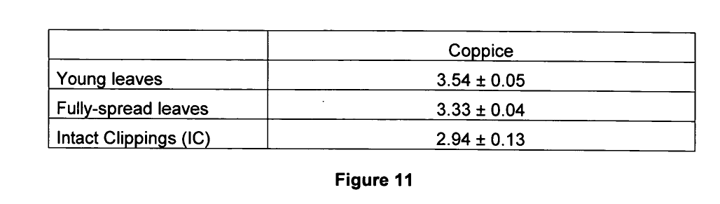

FIG. 12 is a graph illustrating the mean (±s.e.) shikimic acid recoveries from L. styraciflua fruit hulls at different extraction temperatures (mesic habitat, Nacogdoches, Tex., USA) (solvent: DI water) (%, dry wt).

FIG. 13 is table illustrating the recoveries of shikimic acid from L. styraciflua green leaves extracted with room temperature or boiling water (mesic habitat, Nacogdoches, Tex., USA) (solvent: DI water) (%, dry wt)

FIG. 14 is a table illustrating the mean (±s.e.) shikimic acid recoveries of L. styraciflua fruit hulls extracted under different ASE conditions (mesic habitat, Nacogdoches, Tex., USA) (solvent: methanol) (%, dry wt).

FIG. 15 is a flow chart illustrating a simple and fast method for detecting shikimic acid from plant materials.

FIG. 16 is a graph illustrating the mean (±s.e.) shikimic acid recoveries in L. styraciflua green leaves (diamond) and L. styraciflua ‘Texas Star’ yellow leaves (triangle) at different extraction times at room temperature (21-23° C.) (wet habitat, Nacogdoches, Tex., USA) (solvent: DI water) (%, dry wt).

FIG. 17 is a table illustrating the mean (±s.e.) shikimic acid recoveries in L. styraciflua green leaves following agitation over different extraction times at room temperature (wet habitat, Nacogdoches, Tex., USA) (solvent: DI water) (%, dry wt).

FIG. 18 is a graph illustrating the mean (±s.e.) shikimic acid recoveries in L. styraciflua green leaves using different solvent volumes (wet habitat, Nacogdoches, Tex., USA) (solvent: DI water) (%, dry wt).

FIG. 19 is a table illustrating the mean (±s.e.) shikimic acid recoveries from yellow leaves of L. styraciflua ‘Texas Star’ (wet habitat, Nacogdoches, Tex., USA) (solvent: DI water) (%, dry wt).

FIG. 20 is a graph illustrating the elution curve of shikimic acid extract from yellow leaves of L. styraciflua ‘Texas star’ on ion exchange resin (Amberlite IRA-400) column (%, dry wt).

FIG. 21 is a schematic diagram for shikimic acid extraction from Liquidambar tissues.

### DETAILED DESCRIPTION OF THE PREFERRED EMBODIMENT

I. Plant Materials (Specific Examples 4 Through 9):

Although other species, varieties, or cultivars contain shikimic acid, American sweetgum (L. styraciflua L.), particularly the cultivars “Texas Star” (selected by the present inventors) and “rotundiloba,” possess higher shikimic acid content than its Asian counterpart (L. formosana Hance).

The present inventors have determined that leaves, fruits, and annual stems of sweetgum varieties have much higher shikimic acid contents than older stem, bark (both inner and outer), wood, and roots. Extraction experiments identified that young tissues usually contain higher shikimic acid concentration than older tissues, at least during the late growing season. Therefore, high-yield leaf, fruit, and stem tissues of sweetgum are ideal materials for shikimic acid extraction. These materials can be harvested either directly from living or recently felled trees, or even collected from the ground before decomposition, as rain can alter shikimic acid concentrations. Intact clippings of annual stems with leaves demonstrate high shikimic acid content (4%) and are ideal for commercial harvest, particularly from coppice plants or small trees.

Other cultivars of L. styraciflua have similar use in the production of shikimic acid:
1) Liquidambar L. has one species in North America (L. styraciflua L.), two species in eastern Asia (L. formosana Hance and L. acalycina H. T. Chang), and one in Turkey (L. orientalis Mill.).
2) American sweetgum (L. styraciflua L., synonyms: L. gummifera Salisb., L. barbata Stokes, L. macrophylla Oersted, L. styraciflua var. mexicana (L.) Oersted, and L. styraciflua suberosa Schwerin) (Flora of North America Editorial Committee, 1993).
3) Liquidambar styraciflua is often cultivated; a number of cultivars have been introduced into cultivation for their attractive foliage, conical or rounded forms, and dramatic autumn color.
4) Other species of the genus, for example, Liquidambar acalycina (H. T. Chang and Liquidambar orientalis Mill.) have similar use in production of shikimic acid.
5) Altingia Noronha, and Semiliquidambar Hung T. Chang are very close to Liquidambar L. and the three genera have been treated as a subfamily (Altingioideae or Liquidambaroideae) of the Hamamelidaceae, or have elevated the subfamily to the family Altingiaceae (Ickert-Bond et al. 2005). Altingia and Semiliquidambar may also be a very good source for shikimic acid production.
6) Cultivars of Liquidambar styraciflua L.:
   i) “Texas Star’ (new cultivar, patent application in preparation)
   ii) “Rotundiloba’
   iii) ‘Andrew Hewson’
   iv) ‘Anja’
   v) ‘Anneke’
   vi) ‘Aurea’
   vii) ‘Aurora’
   viii) ‘Aureo Marginata’
   ix) ‘Autumn Glow’
   x) ‘Bratzman’
   xi) ‘Burgundy’
   xii) ‘Burgundy Flush’
   xiii) ‘Cherokee’
   xiv) ‘Clydesform’
   xv) ‘Corky’
   xvi) ‘Festeri’
   xvii) ‘Festival’
   xviii) ‘Fremont’
   xix) ‘Globe’
   xx) ‘Goduzam’
   xxi) ‘Golden Treasure’
   xxii) ‘Grazam’
   xxiii) ‘Gum Ball’
   xxiv) ‘Hagen’
   xxv) ‘Jennifer Carol’
   xxvi) ‘Joseph's Coat’
   xxvii) ‘Kia’
   xxviii) ‘Kirsten’
   xxix) ‘Lane Roberts’
   xxx) ‘Levis’
   xxxi) ‘Lollipop’
   xxxii) ‘Manon’
   xxxiii) ‘Midwest Sunset’
   xxxiv) ‘Moonbeam’
   xxxv) ‘Moraine’
   xxxvi) ‘Naree’
   xxxvii) ‘Oconee’
   xxxviii)‘Paarl’
   xxxix) ‘Palo Alto’
   xl) ‘Parasol’
   xli) ‘Pendiloba’
   xlii) ‘Pendula’
   xliii) ‘Penwood’
   xliv) ‘Pieces of Eight’
   xlv) ‘Silver King’
   xivi) ‘Suberosa’
   xlvii) ‘Stared’
   xlviii) ‘Thea’
   xlix) ‘Tirriki’
   l) ‘Variegata’
   li) ‘White Star’
   lii) ‘Worplesdon’;
7) Cultivar of Liquidambar formosana Hance, including ‘Monticola’.

II. Extraction Methods (Specific Examples 2 Through 6):
1. Plant materials extracted by water, including tap water, deionized (DI) water, nanopure water, or any other types of water by methods of soaking, percolation, or any other means for shikimic acid extraction.
2. Water as the solvent for efficient extraction of shikimic acid at temperatures between 15° C. and 85° C., but at room temperature (21-23° C.) is the preferred embodiment.
3. Water as the solvent for efficient extraction of shikimic acid at temperatures below and above room temperature (15-65° C.).
4. Water as the solvent for efficient extraction of shikimic acid over a short time period (five minutes to four hours) or a longer time period (up to five days).
5. Water as the solvent for efficient extraction of shikimic acid at a volume preferably 20 times the plant material weight.
6. Extraction of shikimic acid with water by shaking the water:plant material mixture at a selected temperature range between 15° C.-65° C.
7. The organic solvent ethanol (EtOH) used for extraction of shikimic acid.
8. The organic solvent methanol (MeOH) used for extraction of shikimic acid.

Water is the most economic and environmentally friendly solvent for use in practicing the present methods, but ethanol and methanol may be used on sweetgum plant tissues within the scope of the present invention. When using water, the short extraction time at room or lower temperatures results in time and energy savings and minimal creation of any hazardous waste.

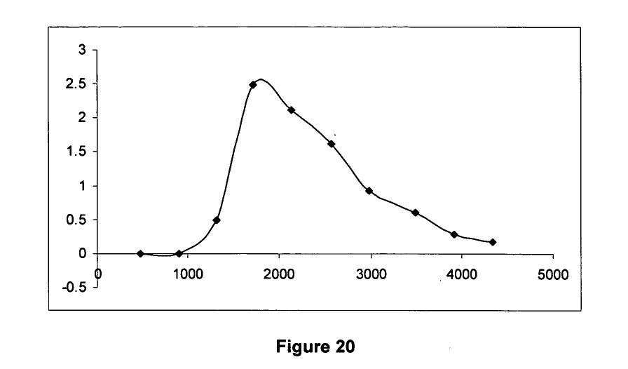

III. Purification Methods (Specific Example 11) (see FIG. 20):
1. Purification of shikimic acid by Diaion HP-20 adsorbent resin chromatography with water elution.
2. Purification of shikimic acid by Amberlite IRA-400 chromatography with acetic acid elution.
3. Purification of shikimic acid by Diaion HP-20 adsorbent resin chromatography with water elution, followed by second purification by Amberlite IRA-400 chromatography with acetic acid elution.

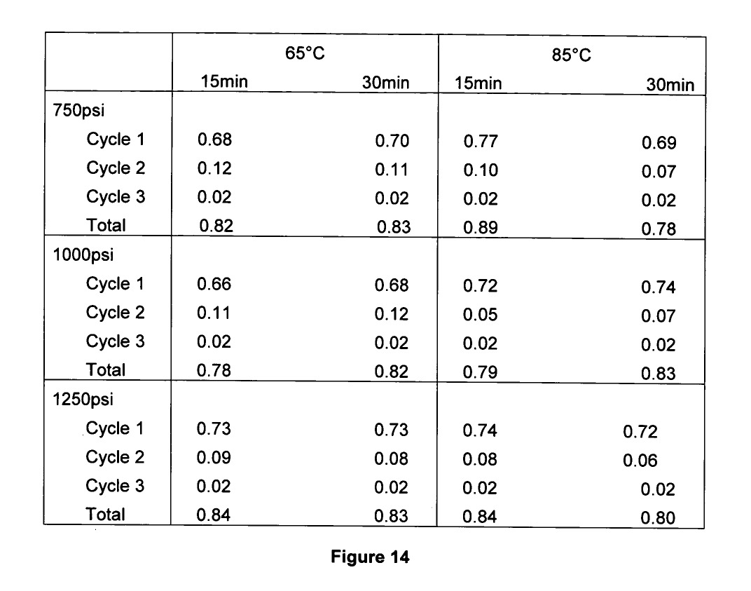

IV. Shikimic Acid Detection Method (Specific Examples 1, 2, 3, and 6) (see FIG. 14):
1. Approximately 1 g of dried, ground plant material.
2. Extraction by ASE 200 extractor (solvent: DI water; preheat: 2 min; heat: 5 min; temperature: 65° C.; pressure: 750 psi; static time: 15 min; flush volume: 60%; purge: 120 s; 2 cycles).
3. HPLC Analysis (column: Zorbax SB—C₁₈, 4.6×250 mm, 5 μm; detection: UV 210 nm; reference: 310 nm; temperature: 36° C.).

V. Specific Examples

### SPECIFIC EXAMPLE 1

### HPLC and NMR Spectral Analysis of Shikimic Acid

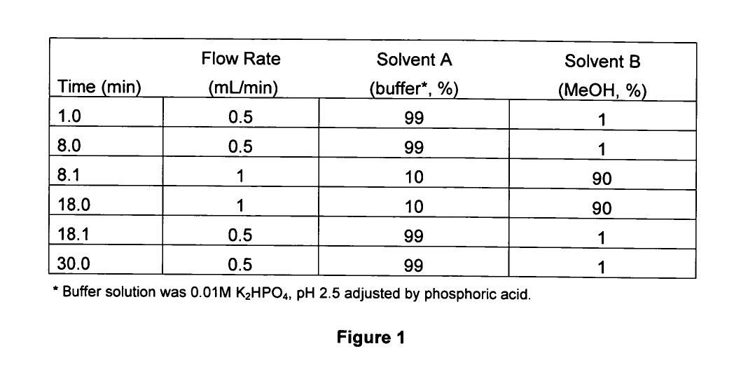

HPLC Analysis: Reagent grade shikimic acid (Acros Organics, Pittsburgh, Pa., USA) was used to prepare a 0.25 mg/mL stock solution in analytical-grade methanol. To determine the calibration curve, a 0.05 mg/mL standard solution was prepared from the stock solution for HPLC (Agilent 1100 Series, Palo Alto, Calif.) analysis (column: Zorbax SB—C₁₈, 4.6×250 mm, 5 μm; mobile phase: (FIG. 1); detection: UV 210 nm, reference 310 nm; temperature: 36° C.).

The calibration curve of standard shikimic acid was investigated between peak area (y) and shikimic acid quantity (x, μg). The calibration equation was y=6848.57671x−0.938134 and the correlation coefficient (γ) was found to be better than 0.9999 for standard shikimic acid in the range of 0.05 to 0.8 μg. Intra- and inter-day accuracy and precision were assessed by conducting five replicated injections of standard shikimic acid. Five injections per day were performed on two consecutive days following sample preparation to determine reproducibility.

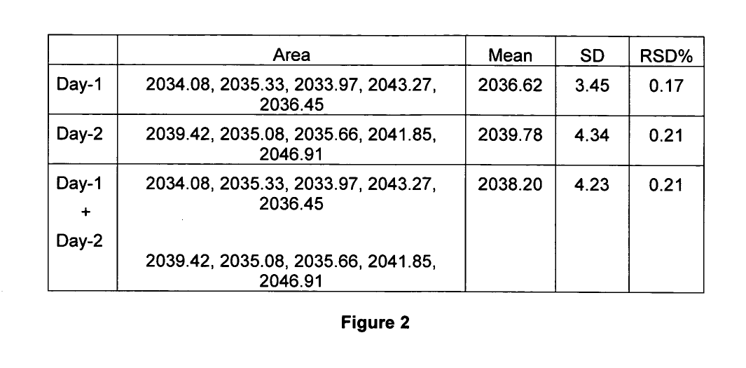

Results are shown in FIG. 2. Standard shikimic acid was used to verify the retention time of 5.3 min. Shikimic acid content of each plant tissue is expressed as a percentage of dry weight (%, dry weight) of plant material.

NMR Analysis: The structures of both standard and laboratory-isolated shikimic acid were confirmed by NMR analysis (Bruker Biospin, 600 MHz NMR) through ¹H, ¹³C, HMQC, and ¹H-¹H COSY experiments. NMR analysis was conducted at The Keck/IMD NMR Center located at The University of Houston, Houston, Tex. NMR data of shikimic acid isolated from L. styraciflua were consistent with those of reagent-grade shikimic acid purchased from Acros Organics and those published by Arisawa et al. (1984).

### SPECIFIC EXAMPLE 2

### Shikimic Acid Extraction Efficacy Following Three Extraction Methods

Plant Materials: Fruit hulls were collected from a mature tree growing in a mesic habitat in Nacogdoches, Texas on Nov. 28, 2005.

Sample Preparation: Plant materials were allowed to air-dry for 24 h and then dried at 65° C. for 24 h in a gravity-flow convection oven (Fisher Scientific, Pittsburgh, Pa.). Dried plant materials were ground using a Thomas-Wiley Mill (Model ED-5, 1 mm openings, Philadelphia, Pa.). Both whole and ground plant materials were deposited as voucher specimens in the National Center for Pharmaceutical Crops, Stephen F. Austin State University, Nacogdoches, Tex.

Extraction: Approximately 1 g of sample was extracted with methanol or DI water with >18 MΩ resistance (B-pure, Barnstead International, Dubuque, Iowa) by one of the following extraction methods: (1) soaking in flasks for 4 h at room temperature (21-23° C.) (with two cycles), (2) Soxhlet extraction using 100 mL of methanol for 4 h at 85° C., or (3) ASE 200 (Accelerated Solvent Extractor, Dionex, Sunnyvale, Calif.) (preheat: 2 min, heat: 5 min, 65° C., 750 psi, 15 min static, 60% volume flush, 120 s purge, and 2 cycles). Samples were loaded into 11 mL cells with diatomaceous earth to prevent sample clumping. Three replications for each treatment were performed for each plant tissue examined.

HPLC Analysis: Method as Presented in Example 1.

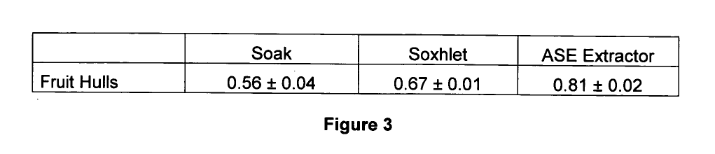

Results and Discussion: Shikimic acid extraction from L. styraciflua fruit hulls was faster and more effective using the ASE when compared to soaking and Soxhlet extraction methods (FIG. 3). The ASE extracted approximately 21% and 46% more shikimic acid than Soxhlet and soaking methods, respectively. Length of extraction time was also significantly shorter using the ASE; a single sample was extracted in 45 min using the ASE while the soaking and Soxhlet extractions lasted 4 h. Utilization of the ASE results in an effective and quick method for shikimic acid extraction. Example 6 demonstrates similar results.

### SPECIFIC EXAMPLE 3

### Shikimic Acid Extraction Efficacy Utilizing Three Different Solvents

Plant Materials: The same fruit hull samples were utilized as presented in Example 2. Yellow leaves were collected from wet habitat in Nacogdoches, Tex. on Dec. 4, 2005.

Sample Preparation: Preparation method as presented in Example 2.

Extraction: (1) Approximately 275 g of yellow leaf sample were soaked for 4 h at room temperature (21-23° C.) (with 3 cycles, see Example 10); (2) ASE extraction method as presented in Example 2.

HPLC Analysis: Method as presented in Example 1.

Results and Discussion: By utilizing the same ASE extraction method as described in Example 2, water extracted approximately 22% and 43% more shikimic acid than methanol and ethanol, respectively. During extraction of shikimic acid with organical solvents, terpenoids (detected at 203 nm by HPLC) were also extracted from sweetgum and made subsequent purification of shikimic acid more difficult (unpublished data by the present inventors). The present inventors have determined water to be an effective and economic solvent for shikimic acid extraction.

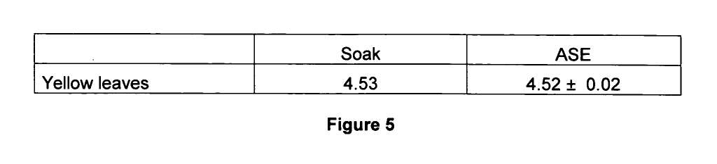

Utilizing water as the solvent results in different extraction methods yielding similar extraction results (FIG. 5). This indicates that water easily extracts shikimic acid so that the effects of different extraction methods are minimal.

### SPECIFIC EXAMPLE 4

### Shikimic Acid Contents in Different Species and Varieties of Liquidambar

Plant Materials: Samples were primarily collected from L. formosana, L. styraciflua, L. styraciflua ‘rotundiloba’, and L. styraciflua ‘Texas Star’ growing in Nacogdoches, Tex. on Nov. 29, 2005. Green and yellow fruits, fruit hulls, green and yellow leaves, stems, and intact clippings (IC) were collected from each species or cultivar. Fruits of L. styraciflua ‘rotundiloba’ were not available because the cultivar is not fertile.

Sample Preparation: Preparation method as presented in Example 2.

Extraction: Samples were extracted by ASE 200 with DI water as solvent (Example 2).

HPLC Analysis: Method as presented in Example 1.

Results and Discussion: American sweetgum (L. styraciflua), including its cultivars ‘rotundiloba’and ‘Texas Star’, have shikimic acid contents 150%-650% higher than their Chinese sibling (L. formosana). ‘Texas Star’ has the highest shikimic acid content among all four tested species and varieties. This cultivar has great potential for the commercial production of shikimic acid.

### SPECIFIC EXAMPLE 5

### Shikimic Acid Contents in Liquidambar styraciflua from Different Habitats

Plant Materials: Samples were collected from three L. styraciflua trees growing in different habitats in Nacogdoches, Tex. between Nov. 29 and Dec. 7, 2005. Green and yellow fruits, fruit hulls, green and yellow leaves, stems, and intact clippings (IC) (annual stems with leaves) were collected from each tree.

Sample Preparation: Preparation method as presented in Example 2.

Extraction: Samples were extracted by ASE 200 with DI water as the solvent (Example 2).

HPLC Analysis: Method as presented in Example 1.

Results and Discussion: Liquidambar styraciflua trees growing in different habitats have sufficient shikimic acid content and can be harvested for shikimic acid production.

### SPECIFIC EXAMPLE 6

### Shikimic Acid Contents in Different Tissues of Liquidambar styraciflua

Plant Materials: Plant samples were collected from a mature tree growing in a dry habitat in Nacogdoches, Tex. on Nov. 27, 2005. The following 15 tissues were collected separately: green fruits, brown fruits (before opening), fruit hulls (without seeds), seeds, green leaves, yellow leaves, whole stem, bud, stem corky wing, intact clippings, stem bark, trunk inner bark, trunk outer bark, root bark, and trunk wood.

Sample Preparation: Preparation method as presented in Example 2.

Extraction: (1) Green fruit, yellow fruit, fruit hull, seed, yellow leaf, stem bark, trunk outer bark, trunk inner bark, and trunk wood samples were homogenized for 30 s. Each tissue was then soaked in 100 mL of methanol for 6 h at room temperature (21-23° C.) (with 2 cycles); (2) Fruit hull and yellow leaf samples were extracted by Soxhlet method with methanol as solvent (100 mL for 4 h); (3) All tissue samples were extracted by ASE 200 with DI water as solvent (see Example 2).

HPLC Analysis: Method as presented in Example 1.

Results and Discussion: The ASE method is consistently more effective in extracting shikimic acid than the soaking and Soxhlet methods. All extraction methods/solvents demonstrate that leaves (green and yellow) and fruits (green and yellow, prior to opening) contain higher shikimic acid concentrations than other tissues. These materials would be significantly useful for commercial shikimic acid extraction and can be easily harvested. Intact clippings have shikimic acid content of approximately 2% and would be ideal for commercial production of shikimic acid considering seasonal availability and the possibility of mechanical harvest. Tissues containing <1% shikimic acid content would not be economically useful for the commercial extraction of shikimic acid. The data also support water as a more effective extraction solvent than ethanol and methanol, as discussed in Example 3.

### SPECIFIC EXAMPLE 7

### Shikimic Acid Contents in Fallen Leaves of Liquidambar styraciflua

Plant Materials: Four types of fallen leaves were collected from trees of L. styraciflua and L. styraciflua ‘Texas Star’ growing in Nacogdoches, Tex. on Dec. 16, 2005. Leaves were categorized as green (harvested from the tree), yellow (harvested from the tree), red (collected from the ground), brown (collected from the ground), or dark brown (collected from the ground).

Sample Preparation: Preparation method as presented in Example 2.

Extraction: Samples were extracted by ASE 200 with DI water as solvent (Example 2).

HPLC Analysis: Method as presented in Example 1.

Results and Discussion: Leaves, particularly green and yellow, that have recently fallen to the ground still contain a high amount of shikimic acid and can be collected for shikimic acid extraction. Fallen red leaves or leaves turning brown before decomposition still had higher shikimic acid content. However, once decomposition has begun and the leaves have darkened, shikimic acid content significantly decreased (0.1%). There is also the possibility of shikimic acid content decreasing in fallen leaves following heavy rain events. Examples 4, 5, 6, 8, and 9 demonstrate that younger tissues contain higher shikimic acid concentrations than older tissues.

### SPECIFIC EXAMPLE 8

### Shikimic Acid Contents in Liquidambar styraciflua Trees Felled During Hurricane Rita

Plant Materials: Green and yellow leaves, and fruit hulls were collected from a living, felled tree (Hurricane Rita, September 2005) in Nacogdoches, Tex. on Dec. 7, 2005. Young leaves and fully-spread leaves were collected from a one-year old coppice around the parent tree.

Sample Preparation: Preparation method as presented in Example 2.

Extraction: Samples were extracted by ASE 200 with DI water as solvent (Example 2).

HPLC Analysis: Method as presented in Example 1.

Results and Discussion: Following root and stem damage (caused by Hurricane Rita), L. styraciflua experienced an induced increase of shikimic acid. Pruning to induce defensive shikimic acid is an effective strategy to increase shikimic acid production in plants.

### SPECIFIC EXAMPLE 9

### Shikimic Acid Contents in Liquidambar styraciflua Coppice Following Prescribed Burning Event

Plant Materials: Following a prescribed burn, L. styraciflua sprouts from the remaining stump or root crown. Young leaves, fully-spread leaves, and intact clippings (IC) were collected from six month old coppice plants in Nacogdoches, Tex. on Dec. 7, 2005.

Sample Preparation: Preparation method as presented in Example 2.

Extraction: Samples were extracted by ASE 200 with DI water as solvent (Example 2).

HPLC Analysis: Method as presented in Example 1.

Results and Discussion: Coppice trees have a higher shikimic acid content and show less variation following a prescribed burn event. Harvesting of intact clippings from smaller coppice trees, as compared to mature trees between 20 to 40 meters in height, is a viable method for obtaining plant materials for commercial extraction of shikimic acid.

### SPECIFIC EXAMPLE 10

### Determination of Optimal Conditions for Utilizing Water as Solvent for Shikimic Acid Extraction from Liquidambar styracilua

Plant Materials: Fruit hulls and green leaf samples were collected from a mature tree growing in a wet habitat in Nacogdoches, Tex. on Nov. 28 and Dec. 4, 2005, respectively.

Sample Preparation: Preparation method as presented in Example 2.

Extraction:

1) Extraction Temperature Analysis:

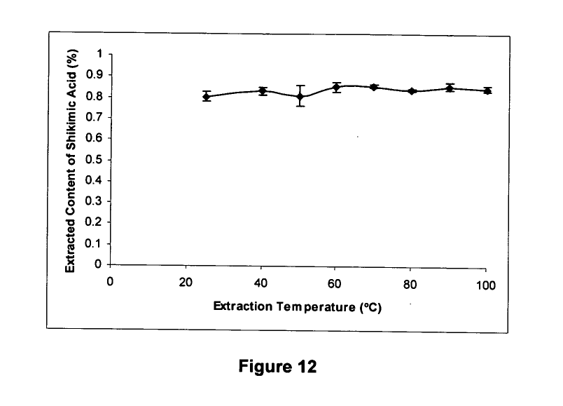

a. Approximately 1 g of L. styraciflua fruit hull sample was extracted by ASE 200 over a temperature gradient of 25, 40, 50, 60, 70, 80, 90, and 100° C. Other extraction parameters were kept the same (solvent: DI water, preheat: 2 min, heat: 5 min, 750 psi, 15 min static time, 60% volume flush, 120 s purge, and 2 cycles) (FIG. 12). Three replications were performed at each temperature. Extractions were analyzed by HPLC for shikimic acid content.

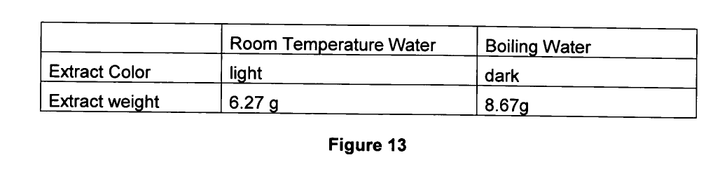

b. Approximately 20 g of L. styraciflua green leaf sample were soaked with 300 mL at room temperature or 100° C. for 1 h with three cycles. The extracts dried with evaporator were weighed (FIG. 13).

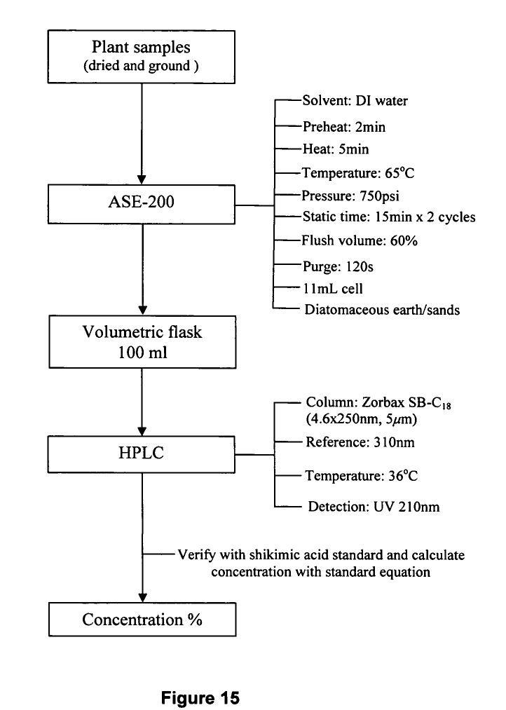

2) Extraction Method Analysis: The effects of pressure, temperature, and static-extraction time on shikimic acid extraction were co-investigated. Approximately 1 g of L. styraciflua dried fruit hull was extracted by ASE 200 with DI water as the solvent (preheat: 2 min, heat: 5 min, 60% volume flush, 120 s purge) (FIG. 14). Extractions were analyzed by HPLC for shikimic acid content. The schematic diagram of the optimum method is presented in FIG. 15.

3) Extraction Time Analysis:

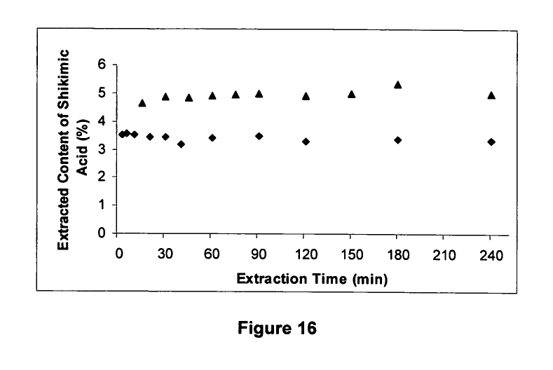

a. Approximately 1 g of L. styraciflua green leaf sample was soaked in 250 mL of DI water for 2, 5, 10, 20, 30, 40, 60, 90, 120, 180, or 240 min at room temperature (21-23° C.) (FIG. 16). Samples were manually shaken for 5 s. Following filtration, solutions were analyzed by HPLC for shikimic acid content.

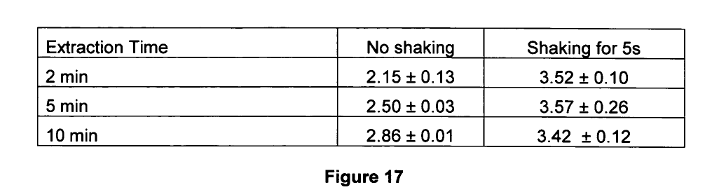

b. Approximately 1 g of L. styraciflua ‘Texas Star’ yellow leaf sample was soaked in 250 mL of DI water for 2, 5, or 10 min at room temperature (21-23° C.) (FIG. 17). Two replicates were performed for each time period. One set of samples was manually shaken for 5 s and another set was not shaken. Following filtration, solutions were analyzed by HPLC for shikimic acid content.

c. Approximately 275 g of L. styraciflua ‘Texas Star’ yellow leaf sample were soaked in 2.75 L of DI water for 15, 30, 45, 60, 75, 90, 120, 180, or 240 min at room temperature (21-23° C.) (FIG. 16). Following filtration, solutions were analyzed by HPLC for shikimic acid content.

4) Solvent Volume Analysis:

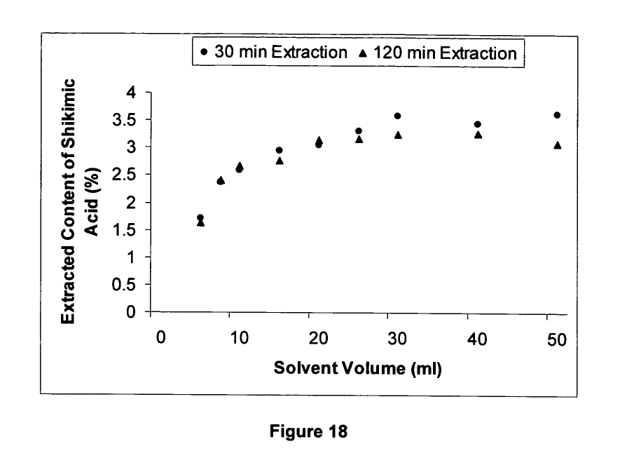

Approximately 1 g of L. styraciflua green leaf sample was percolated with different volumes of DI water (5, 7.5, 10, 15, 20, 25, 30, 40, or 50 mL) for 30 or 120 min (FIG. 18). Following filtration, solutions were analyzed by HPLC for shikimic acid content.

HPLC Analysis: Method as presented in Example 1.

FIG. 12 Results: With water as the solvent, extraction temperature has minimal effect on recovery of shikimic acid.

FIG. 14 Results: With water as the solvent, there was no significant difference in shikimic acid extract concentrations in L. styraciflua fruit hulls among the different extraction conditions investigated. The first two cycles extracted approximately 97.6% of the total shikimic acid. To decrease extraction time, we chose the first two cycles to represent the content of shikimic acid in L. styraciflua tissues.

FIG. 16 Results: Shikimic acid can be easily extracted with water. Length of extraction time had no significant difference on shikimic acid extract concentrations. For commercial production, short time of extraction (e.g., several minutes) is cost-effective in terms of both time and energy.

FIG. 17 Results: Although length of extraction time had no significant difference on shikimic acid extract concentrations, shaking or stirring the solution can improve extraction efficacy by up to 50%.

FIG. 18 Results: To efficiently extract shikimic acid from sweetgum, preferably 20 times of water is to be used (volume of water mL: weight of plant weight g=20:1). Higher extraction efficacy can be obtained if the material is extracted in two cycles rather than in one with the same amount of water. The data also demonstrate that shikimic acid extract concentration following a 30 min extraction time is not significantly different after 2 h of extraction.

Conclusion for Specific Example 10: Shikimic acid in L. styraciflua tissues can be easily and quickly extracted using water at room temperature. The experiments demonstrated that shikimic acid can be almost fully extracted in 5 min at room temperature. Temperature also had no significant impact on extraction efficiency. However, as the extraction temperature is increased, more compounds in addition to shikimic acid are extracted, which causes the purification of shikimic acid to become more difficult. Water volume demonstrated no significant impact on extracted shikimic acid content (%).

### SPECIFIC EXAMPLE 11

### Shikimic Acid Extraction and Purification from Liquidambar styraciflua

Plant Materials: Yellow leaves of L. styraciflua ‘Texas Star’ were collected from a wet habitat in Nacogdoches, Tex. on Dec. 4, 2005.

Sample Preparation: Preparation method as presented in Example 2.

Extraction: Approximately 275 g of L. styraciflua ‘Texas Star’ yellow leaf sample were soaked in 2.75 L of DI water for 4 h, with two additional extraction cycles (4 h each).

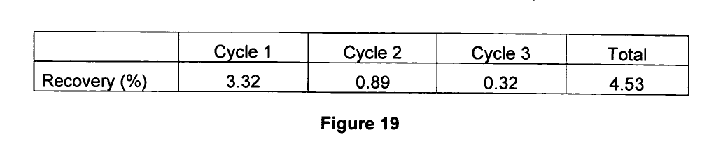

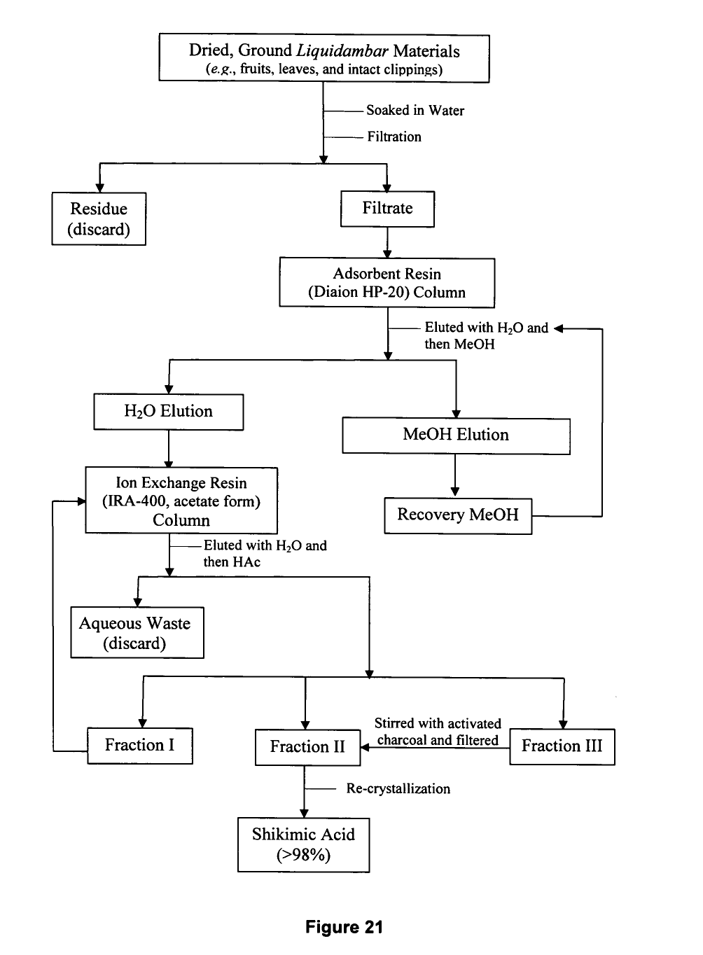

Purification: Following filtration, 5 mL of extract from each cycle was collected for HPLC analysis (see method in Example 1). Remaining extract was passed through a Diaion HP-20 adsorbent resin column and eluted with DI water. The collected solution was then passed through a column of Amberlite IRA-400 ion exchange resin (acetate form), and the column washed with DI water. Shikimic acid was not detected in the collected aqueous elution. The column was then eluted with 4 L 25% acetic acid to give three fractions (Fraction I 800˜1200 mL, Fraction II 1200˜1600 mL, Fraction III 1600˜4000 mL). Each fraction was evaporated with rotary evaporator under reduced pressure to give three residues (1, 2, and 3). Residue 1 was resolved in water and re-passed through the ion exchange chromatography column. The combined elution, including shikimic acid, was evaporated to give residue 4. Residue 3 was resolved in methanol, decolorized with activated charcoal, and then filtered. The filtered solution, together with residue 2 and 4, was combined and then recrystallized in a mixture of ethyl acetate and methanol to afford purified shikimic acid (8.1 g, 98%) (FIGS. 19-21).

Results: This very efficient and economic method with minimal generation of hazardous waste can be scaled up for commercial production of shikimic acid from L. styraciflua.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention.

It is, therefore, contemplated that the appended claims will cover such modifications that fall within the scope of the invention.

We claim:

1. A method for the production of shikimic acid comprising the steps of:
selecting a quantum of sweetgum plant tissue; and
performing an extraction process on said sweetgum plant tissue as a substrate; and
collecting shikimic acid produced by said extraction process.

2. The method of claim 1 wherein water is used as a solvent in said extraction process.

3. The method of claim 1 wherein ethanol is used as a solvent in said extraction process.

4. The method of claim 1 wherein methanol is used as a solvent in said extraction process.

5. The method of claim 1 wherein an organic solvent is used as a solvent in said extraction process.

6. The method of claim 1 wherein solvents used in said extraction process consist essentially of water.

7. The method of claim 1 wherein said sweetgum plant tissue is selected substantially from a group consisting of sweetgum plant leaves and sweetgum fruit.

8. The method of claim 1 wherein said sweetgum plant tissue is harvested substantially from one or more plants chosen from a group consisting of:
L. styraciflua L.;
L. styraciflua L. cultivar ‘Texas Star’;
L. styraciflua L. cultivar ‘rotundiloba’;
L. formosana Hance;
L. acalycina H. T. Chang;
L. orientalis Mill;
L. gummifera Salisb.;
L. barbata Stokes;
L. macrophylla Oersted;
L. styraciflua var. mexicana (L.) Oersted;
L. styraciflua suberosa Schwerin;
Altingia Noronha;
Semiliquidambar (H. T. Chang);
Altingiaceae (Ickert-Bond et al., 2005); and
Cultivars of Liquidambar styraciflua L. comprising:
‘Texas Star’ (new cultivar, patent application in preparation)
‘Rotundiloba’
‘Andrew Hewson’
‘Anja’
‘Anneke’
‘Aurea’
‘Aurora’
‘Aureo Marginata’
‘Autumn Glow’
‘Bratzman’
‘Burgundy’
‘Burgundy Flush’
‘Cherokee’
‘Clydesform’
‘Corky’
‘Festeri’
‘Festival’
‘Fremont’
‘Globe’
‘Goduzam’
‘Golden Treasure’
‘Grazam’
‘Gum Ball’
‘Hagen’
‘Jennifer Carol’
‘Joseph's Coat’
‘Kia’
‘Kirsten’
‘Lane Roberts’
‘Levis’
‘Lollipop’
‘Manon’
‘Midwest Sunset’
‘Moonbeam’
‘Moraine’
‘Naree’
‘Oconee’
‘Paarl’
‘Palo Alto’
‘Parasol’
‘Pendiloba’
‘Pendula’
‘Penwood’
‘Pieces of Eight’
‘Silver King’
‘Suberosa’
‘Stared’
‘Thea’
‘Tirriki’
‘Variegata’
‘White Star’
‘Worplesdon’; and
Cultivars of Liquidambar formosana Hance.

9. The method of claim 2 wherein said sweetgum plant tissue is harvested substantially from one or more plants chosen from a group consisting of:
L. styraciflua L.;
L. styraciflua L. cultivar ‘Texas Star’;
L. styraciflua L. cultivar ‘rotundiloba’;
L. formosana Hance;
L. acalycina H. T. Chang;
L. orientalis Mill;
L. gummifera Salisb.;
L. barbata Stokes;
L. macrophylla Oersted;
L. styraciflua var. mexicana (L.) Oersted;
L. styraciflua suberosa Schwerin;
Altingia Noronha;
Semiliquidambar (H. T. Chang);
Altingiaceae (Ickert-Bond et al. 2005); and
Cultivars of Liquidambar styraciflua L. comprising:
‘Texas Star’ (new cultivar, patent application in preparation)
‘Rotundiloba’
‘Andrew Hewson’
‘Anja’
‘Anneke’
‘Aurea’
‘Aurora’
‘Aureo Marginata’
‘Autumn Glow’
‘Bratzman’
‘Burgundy’
‘Burgundy Flush’
‘Cherokee’
‘Clydesform’
‘Corky’
‘Festeri’
‘Festival’
‘Fremont’
‘Globe’
‘Goduzam’
‘Golden Treasure’
‘Grazam’
‘Gum Ball’
‘Hagen’
‘Jennifer Carol’
‘Joseph's Coat’
‘Kia’
‘Kirsten’
‘Lane Roberts’
‘Levis’
‘Lollipop’
‘Manon’
‘Midwest Sunset’
‘Moonbeam’
‘Moraine’
‘Naree’
‘Oconee’
‘Paarl’
‘Palo Alto’
‘Parasol’
‘Pendiloba’
‘Pendula’
‘Penwood’
‘Pieces of Eight’
‘Silver King’
‘Suberosa’
‘Stared’
‘Thea’
‘Tirriki’
‘Variegata’
‘White Star’
‘Worplesdon’; and
Cultivars of Liquidambar formosana Hance.

10. The method of claim 3 wherein said sweetgum plant tissue is harvested substantially from one or more plants chosen from a group consisting of:
L. styraciflua L.;
L. styraciflua L. cultivar ‘Texas Star’;
L. styraciflua L. cultivar ‘rotundiloba’;
L. formosana Hance;
L. acalycina H. T. Chang;
L. orientalis Mill;
L. gummifera Salisb.;
L. barbata Stokes;
L. macrophylla Oersted;
L. styraciflua var. mexicana (L.) Oersted;
L. styraciflua suberosa Schwerin;
Altingia Noronha;
Semiliquidambar (H. T. Chang);
Altingiaceae (Ickert-Bond et al. 2005); and
Cultivars of Liquidambar styraciflua L. comprising:
‘Texas Star’ (new cultivar, patent application in preparation)
‘Rotundiloba’
‘Andrew Hewson’
‘Anja’
‘Anneke’
‘Aurea’
‘Aurora’
‘Aureo Marginata’
‘Autumn Glow’
‘Bratzman’
‘Burgundy’
‘Burgundy Flush’
‘Cherokee’
‘Clydesform’
‘Corky’
‘Festeri’
‘Festival’
‘Fremont’
‘Globe’
‘Goduzam’
‘Golden Treasure’
‘Grazam’
‘Gum Ball’
‘Hagen’
‘Jennifer Carol’
‘Joseph's Coat’
‘Kia’
‘Kirsten’
‘Lane Roberts’
‘Levis’
‘Lollipop’
‘Manon’
‘Midwest Sunset’
‘Moonbeam’
‘Moraine’
‘Naree’
‘Oconee’
‘Paarl’
‘Palo Alto’
‘Parasol’
‘Pendiloba’
‘Pendula’
‘Penwood’
‘Pieces of Eight’
‘Silver King’
‘Suberosa’
‘Stared’
‘Thea’
‘Tirriki’
‘Variegata’
‘White Star’
‘Worplesdon’; and
Cultivars of Liquidambar formosana Hance.

11. The method of claim 4 wherein said sweetgum plant tissue is harvested substantially from one or more plants chosen from a group consisting of:
L. styraciflua L.;
L. styraciflua L. cultivar ‘Texas Star’;
L. styraciflua L. cultivar ‘rotundiloba’;
L. formosana Hance;
L. acalycina H. T. Chang;
L. orientalis Mill;
L. gummifera Salisb.;
L. barbata Stokes;
L. macrophylla Oersted;
L. styraciflua var. mexicana (L.) Oersted;
L. styraciflua suberosa Schwerin;
Altingia Noronha;
Semiliquidambar (H. T. Chang);
Altingiaceae (Ickert-Bond et al. 2005); and
Cultivars of Liquidambar styraciflua L. comprising:
‘Texas Star’ (new cultivar, patent application in preparation)
‘Rotundiloba’
‘Andrew Hewson’
‘Anja’
‘Anneke’
‘Aurea’
‘Aurora’
‘Aureo Marginata’
‘Autumn Glow’
‘Bratzman’
‘Burgundy’
‘Burgundy Flush’
‘Cherokee’
‘Clydesform’
‘Corky’
‘Festeri’
‘Festival’
‘Fremont’
‘Globe’
‘Goduzam’
‘Golden Treasure’
‘Grazam’
‘Gum Ball’
‘Hagen’
‘Jennifer Carol’
‘Joseph's Coat’
‘Kia’
‘Kirsten’
‘Lane Roberts’
‘Levis’
‘Lollipop’
‘Manon’
‘Midwest Sunset’
‘Moonbeam’
‘Moraine’
‘Naree’
‘Oconee’
‘Paarl’
‘Palo Alto’
‘Parasol’
‘Pendiloba’
‘Pendula’
‘Penwood’
‘Pieces of Eight’
‘Silver King’
‘Suberosa’
‘Stared’
‘Thea’
‘Tirriki’
‘Variegata’
‘White Star’
‘Worplesdon’; and
Cultivars of Liquidambar formosana Hance.

12. The method of claim 5 wherein said sweetgum plant tissue is harvested substantially from one or more plants chosen from a group consisting of:
L. styraciflua L.;
L. styraciflua L. cultivar ‘Texas Star’;
L. styraciflua L. cultivar ‘rotundiloba’;
L. formosana Hance;
L. acalycina H. T. Chang;
L. orientalis Mill;
L. gummifera Salisb.;
L. barbata Stokes;
L. macrophylla Oersted;
L. styraciflua var. mexicana (L.) Oersted;
L. styraciflua suberosa Schwerin;
Altingia Noronha;
Semiliquidambar (H. T. Chang);
Altingiaceae (Ickert-Bond et al. 2005); and
Cultivars of Liquidambar styraciflua L. comprising:
‘Texas Star’ (new cultivar, patent application in preparation)
‘Rotundiloba’
‘Andrew Hewson’
‘Anja’
‘Anneke’
‘Aurea’
‘Aurora’
‘Aureo Marginata’
‘Autumn Glow’
‘Bratzman’
‘Burgundy’
‘Burgundy Flush’
‘Cherokee’
‘Clydesform’
‘Corky’
‘Festeri’
‘Festival’
‘Fremont’
‘Globe’
‘Goduzam’
‘Golden Treasure’
‘Grazam’
‘Gum Ball’
‘Hagen’
‘Jennifer Carol’
‘Joseph's Coat’
‘Kia’
‘Kirsten’
‘Lane Roberts’
‘Levis’
‘Lollipop’
‘Manon’
‘Midwest Sunset’
‘Moonbeam’
‘Moraine’
‘Naree’
‘Oconee’
‘Paarl’
‘Palo Alto’
‘Parasol’
‘Pendiloba’
‘Pendula’
‘Penwood’
‘Pieces of Eight’
‘Silver King’
‘Suberosa’
‘Stared’
‘Thea’
‘Tirriki’
‘Variegata’
‘White Star’
‘Worplesdon’; and
Cultivars of Liquidambar formosana Hance.

13. The method of claim 1 further comprising the step, after said extraction, of purification of shikimic acid by adsorbent resin chromatography with water elution.

14. The method of claim 1 further comprising the step, after said extraction, of purification of shikimic acid by chromatography with acetic acid elution.

15. The method of claim 1 further comprising the step, after said extraction, of first purification of shikimic acid by adsorbent resin chromatography with water elution, followed by second purification by chromatography with acetic acid elution.

16. The method of claim 2 further comprising the step, after said extraction, of purification of shikimic acid by adsorbent resin chromatography with water elution.

17. The method of claim 2 further comprising the step, after said extraction, of purification of shikimic acid by chromatography with acetic acid elution.

18. The method of claim 2 further comprising the step, after said extraction, of first purification of shikimic acid by adsorbent resin chromatography with water elution, followed by second purification by chromatography with acetic acid elution.

19. The method of claim 5 further comprising the step, after said extraction, of purification of shikimic acid by adsorbent resin chromatography with water elution.

20. The method of claim 5 further comprising the step, after said extraction, of purification of shikimic acid by chromatography with acetic acid elution.

21. The method of claim 5 further comprising the step, after said extraction, of first purification of shikimic acid by adsorbent resin chromatography with water elution, followed by second purification by chromatography with acetic acid elution.

22. The method of claim 7 further comprising the step, after said extraction, of purification of shikimic acid by adsorbent resin chromatography with water elution.

23. The method of claim 7 further comprising the step, after said extraction, of purification of shikimic acid by chromatography with acetic acid elution.

24. The method of claim 7 further comprising the step, after said extraction, of first purification of shikimic acid by adsorbent resin chromatography with water elution, followed by second purification by chromatography with acetic acid elution.

25. The method of claim 8 further comprising the step, after said extraction, of purification of shikimic acid by adsorbent resin chromatography with water elution.

26. The method of claim 8 further comprising the step, after said extraction, of purification of shikimic acid by chromatography with acetic acid elution.

27. The method of claim 8 further comprising the step, after said extraction, of first purification of shikimic acid by adsorbent resin chromatography with water elution, followed by second purification by chromatography with acetic acid elution.

28. The method of claim 1 wherein said sweetgum plant tissue is harvested substantially from one or more plants in the plant group of L. styraciflua L.

29. The method of claim 28 further comprising the step, after said extraction, of purification of shikimic acid by adsorbent resin chromatography with water elution.

30. The method of claim 28 further comprising the step, after said extraction, of purification of shikimic acid by chromatography with acetic acid elution.

31. The method of claim 28 further comprising the step, after said extraction, of first purification of shikimic acid by adsorbent resin chromatography with water elution, followed by second purification by chromatography with acetic acid elution.

32. Shikimic acid produced by process steps comprising:
selecting a quantum of sweetgum plant tissue; and
performing an extraction process on said sweetgum plant tissue as a substrate; and
collecting shikimic acid produced by said extraction process.

33. The method of claim 32 wherein water is used as a solvent in said extraction process.

34. The method of claim 32 wherein ethanol is used as a solvent in said extraction process.

35. The method of claim 32 wherein methanol is used as a solvent in said extraction process.

36. The method of claim 32 wherein an organic solvent is used as a solvent in said extraction process.

37. The method of claim 32 wherein solvents used in said extraction process consist essentially of water.

38. The method of claim 32 wherein said sweetgum plant tissue is selected substantially from a group consisting of sweetgum plant leaves and sweetgum fruit.

39. The method of claim 32 wherein said sweetgum plant tissue is harvested substantially from one or more plants chosen from a group consisting of:
L. styraciflua L.;
L. styraciflua L. cultivar ‘Texas Star’;
L. styraciflua L. cultivar ‘rotundiloba’;
L. formosana Hance;
L. acalycina H. T. Chang;
L. orientalis Mill;
L. gummifera Salisb.;
L. barbata Stokes;
L. macrophylla Oersted;
L. styraciflua var. mexicana (L.) Oersted;
L. styraciflua suberosa Schwerin;
Altingia Noronha;
Semiliquidambar (H. T. Chang);
Altingiaceae (Ickert-Bond et al. 2005); and
Cultivars of Liquidambar styraciflua L. comprising:
‘Texas Star’ (new cultivar, patent application in preparation)
‘Rotundiloba’
‘Andrew Hewson’
‘Anja’
‘Anneke’
‘Aurea’
‘Aurora’
‘Aureo Marginata’
‘Autumn Glow’
‘Bratzman’
‘Burgundy’
‘Burgundy Flush’
‘Cherokee’
‘Clydesform’
‘Corky’
‘Festeri’
‘Festival’
‘Fremont’
‘Globe’
‘Goduzam’
‘Golden Treasure’
‘Grazam’
‘Gum Ball’
‘Hagen’
‘Jennifer Carol’
‘Joseph's Coat’
‘Kia’
‘Kirsten’
‘Lane Roberts’
‘Levis’
‘Lollipop’
‘Manon’
‘Midwest Sunset’
‘Moonbeam’
‘Moraine’
‘Naree’
‘Oconee’
‘Paarl’
‘Palo Alto’
‘Parasol’
‘Pendiloba’
‘Pendula’
‘Penwood’
‘Pieces of Eight’
‘Silver King’
‘Suberosa’
‘Stared’
‘Thea’
‘Tirriki’
‘Variegata’
‘White Star’
‘Worplesdon’; and
Cultivars of Liquidambar formosana Hance.

40. The method of claim 33 wherein said sweetgum plant tissue is harvested substantially from one or more plants chosen from a group consisting of:
L. styraciflua L.;
L. styraciflua L. cultivar ‘Texas Star’;
L. styraciflua L. cultivar ‘rotundiloba’;
L. formosana Hance;
L. acalycina H. T. Chang;
L. orientalis Mill;
L. gummifera Salisb.;
L. barbata Stokes;
L. macrophylla Oersted;
L. styraciflua var. mexicana (L.) Oersted;
L. styraciflua suberosa Schwerin;
Altingia Noronha;
Semiliquidambar (H. T. Chang);
Altingiaceae (Ickert-Bond et al. 2005); and
Cultivars of Liquidambar styraciflua L. comprising:
‘Texas Star’ (new cultivar, patent application in preparation)
‘Rotundiloba’
‘Andrew Hewson’
‘Anja’
‘Anneke’
‘Aurea’
‘Aurora’
‘Aureo Marginata’
‘Autumn Glow’
‘Bratzman’
‘Burgundy’
‘Burgundy Flush’
‘Cherokee’
‘Clydesform’
‘Corky’
‘Festeri’
‘Festival’
‘Fremont’
‘Globe’
‘Goduzam’
‘Golden Treasure’
‘Grazam’
‘Gum Ball’
‘Hagen’
‘Jennifer Carol’
‘Joseph's Coat’
‘Kia’
‘Kirsten’
‘Lane Roberts’
‘Levis’
‘Lollipop’
‘Manon’
‘Midwest Sunset’
‘Moonbeam’
‘Moraine’
‘Naree’
‘Oconee’
‘Paarl’
‘Palo Alto’
‘Parasol’
‘Pendiloba’
‘Pendula’
‘Penwood’
‘Pieces of Eight’
‘Silver King’
‘Suberosa’
‘Stared’
‘Thea’
‘Tirriki’
‘Variegata’
‘White Star’
‘Worplesdon’; and
Cultivars of Liquidambar formosana Hance.

41. The method of claim 34 wherein said sweetgum plant tissue is harvested substantially from one or more plants chosen from a group consisting of:
L. styraciflua L.;
L. styraciflua L. cultivar ‘Texas Star’;
L. styraciflua L. cultivar ‘rotundiloba’;
L. formosana Hance;
L. acalycina H. T. Chang;
L. orientalis Mill;
L. gummifera Salisb.;
L. barbata Stokes;
L. macrophylla Oersted;
L. styraciflua var. mexicana (L.) Oersted;
L. styraciflua suberosa Schwerin;
Altingia Noronha;
Semiliquidambar (H. T. Chang);
Altingiaceae (Ickert-Bond et al. 2005); and
Cultivars of Liquidambar styraciflua L. comprising:
‘Texas Star’ (new cultivar, patent application in preparation)
‘Rotundiloba’
‘Andrew Hewson’
‘Anja’
‘Anneke’
‘Aurea’
‘Aurora’
‘Aureo Marginata’
‘Autumn Glow’
‘Bratzman’
‘Burgundy’
‘Burgundy Flush’
‘Cherokee’
‘Clydesform’
‘Corky’
‘Festeri’
‘Festival’
‘Fremont’
‘Globe’
‘Goduzam’
‘Golden Treasure’
‘Grazam’
‘Gum Ball’
‘Hagen’
‘Jennifer Carol’
‘Joseph's Coat’
‘Kia’
‘Kirsten’
‘Lane Roberts’
‘Levis’
‘Lollipop’
‘Manon’
‘Midwest Sunset’
‘Moonbeam’
‘Moraine’
‘Naree’
‘Oconee’
‘Paarl’
‘Palo Alto’
‘Parasol’
‘Pendiloba’
‘Pendula’
‘Penwood’
‘Pieces of Eight’
‘Silver King’
‘Suberosa’
‘Stared’
‘Thea’
‘Tirriki’
‘Variegata’
‘White Star’
‘Worplesdon’; and
Cultivars of Liquidambar formosana Hance.

42. The method of claim 35 wherein said sweetgum plant tissue is harvested substantially from one or more plants chosen from a group consisting of:
L. styraciflua L.;
L. styraciflua L. cultivar ‘Texas Star’;
L. styraciflua L. cultivar ‘rotundiloba’;
L. formosana Hance;
L. acalycina H. T. Chang;
L. orientalis Mill;
L. gummifera Salisb.;
L. barbata Stokes;
L. macrophylla Oersted;
L. styraciflua var. mexicana (L.) Oersted;
L. styraciflua suberosa Schwerin;
Altingia Noronha;
Semiliquidambar (H. T. Chang);
Altingiaceae (Ickert-Bond et al. 2005); and
Cultivars of Liquidambar styraciflua L. comprising:
‘Texas Star’ (new cultivar, patent application in preparation)
‘Rotundiloba’
‘Andrew Hewson’
‘Anja’
‘Anneke’
‘Aurea’
‘Aurora’
‘Aureo Marginata’
‘Autumn Glow’
‘Bratzman’
‘Burgundy’
‘Burgundy Flush’
‘Cherokee’
‘Clydesform’
‘Corky’
‘Festeri’
‘Festival’
‘Fremont’
‘Globe’
‘Goduzam’
‘Golden Treasure’
‘Grazam’
‘Gum Ball’
‘Hagen’
‘Jennifer Carol’
‘Joseph's Coat’
‘Kia’
‘Kirsten’
‘Lane Roberts’
‘Levis’
‘Lollipop’
‘Manon’
‘Midwest Sunset’
‘Moonbeam’
‘Moraine’
‘Naree’
‘Oconee’
‘Paarl’
‘Palo Alto’
‘Parasol’
‘Pendiloba’
‘Pendula’
‘Penwood’
‘Pieces of Eight’
‘Silver King’
‘Suberosa’
‘Stared’
‘Thea’
‘Tirriki’
‘Variegata’
‘White Star’
‘Worplesdon’; and
Cultivars of Liquidambar formosana Hance.

43. The method of claim 36 wherein said sweetgum plant tissue is harvested substantially from one or more plants chosen from a group consisting of:
L. styraciflua L.;
L. styraciflua L. cultivar ‘Texas Star’;
L. styraciflua L. cultivar ‘rotundiloba’;
L. formosana Hance;
L. acalycina H. T. Chang;
L. orientalis Mill;
L. gummifera Salisb.;
L. barbata Stokes;
L. macrophylla Oersted;
L. styraciflua var. mexicana (L.) Oersted;
L. styraciflua suberosa Schwerin;
Altingia Noronha;
Semiliquidambar (H. T. Chang);
Altingiaceae (Ickert-Bond et al. 2005); and
Cultivars of Liquidambar styraciflua L. comprising:
‘Texas Star’ (new cultivar, patent application in preparation)
‘Rotundiloba’
‘Andrew Hewson’
‘Anja’
‘Anneke’
‘Aurea’
‘Aurora’
‘Aureo Marginata’
‘Autumn Glow’
‘Bratzman’
‘Burgundy’
‘Burgundy Flush’
‘Cherokee’
‘Clydesform’
‘Corky’
‘Festeri’
‘Festival’
‘Fremont’
‘Globe’
‘Goduzam’
‘Golden Treasure’
‘Grazam’
‘Gum Ball’
‘Hagen’
‘Jennifer Carol’
‘Joseph's Coat’
‘Kia’
‘Kirsten’
‘Lane Roberts’
‘Levis’
‘Lollipop’
‘Manon’
‘Midwest Sunset’
‘Moonbeam’
‘Moraine’
‘Naree’
‘Oconee’
‘Paarl’
‘Palo Alto’
‘Parasol’
‘Pendiloba’
‘Pendula’
‘Penwood’
‘Pieces of Eight’
‘Silver King’
‘Suberosa’
‘Stared’
‘Thea’
‘Tirriki’
‘Variegata’
‘White Star’
‘Worplesdon’; and
Cultivars of Liquidambar formosana Hance.

44. The method of claim 32 further comprising the step, after said extraction, of purification of shikimic acid by adsorbent resin chromatography with water elution.

45. The method of claim 32 further comprising the step, after said extraction, of purification of shikimic acid by chromatography with acetic acid elution.

46. The method of claim 32 further comprising the step, after said extraction, of first purification of shikimic acid by adsorbent resin chromatography with water elution, followed by second purification by chromatography with acetic acid elution.

47. The method of claim 33 further comprising the step, after said extraction, of purification of shikimic acid by adsorbent resin chromatography with water elution.

48. The method of claim 33 further comprising the step, after said extraction, of purification of shikimic acid by chromatography with acetic acid elution.

49. The method of claim 33 further comprising the step, after said extraction, of first purification of shikimic acid by adsorbent resin chromatography with water elution, followed by second purification by chromatography with acetic acid elution.

50. The method of claim 36 further comprising the step, after said extraction, of purification of shikimic acid by adsorbent resin chromatography with water elution.

51. The method of claim 36 further comprising the step, after said extraction, of purification of shikimic acid by chromatography with acetic acid elution.

52. The method of claim 36 further comprising the step, after said extraction, of first purification of shikimic acid by adsorbent resin chromatography with water elution, followed by second purification by chromatography with acetic acid elution.

52. The method of claim 38 further comprising the step, after said extraction, of purification of shikimic acid by adsorbent resin chromatography with water elution.

53. The method of claim 38 further comprising the step, after said extraction, of purification of shikimic acid by chromatography with acetic acid elution.

54. The method of claim 38 further comprising the step, after said extraction, of first purification of shikimic acid by adsorbent resin chromatography with water elution, followed by second purification by chromatography with acetic acid elution.

55. The method of claim 39 further comprising the step, after said extraction, of purification of shikimic acid by adsorbent resin chromatography with water elution.

56. The method of claim 39 further comprising the step, after said extraction, of purification of shikimic acid by chromatography with acetic acid elution.

57. The method of claim 39 further comprising the step, after said extraction, of first purification of shikimic acid by adsorbent resin chromatography with water elution, followed by second purification by chromatography with acetic acid elution.

58. The method of claim 32 wherein said sweetgum plant tissue is harvested substantially from one or more plants in the plant group of L. styraciflua L.

59. The method of claim 28 further comprising the step, after said extraction, of purification of shikimic acid by adsorbent resin chromatography with water elution.

60. The method of claim 28 further comprising the step, after said extraction, of purification of shikimic acid by chromatography with acetic acid elution.

61. The method of claim 28 further comprising the step, after said extraction, of first purification of shikimic acid by adsorbent resin chromatography with water elution, followed by second purification by chromatography with acetic acid elution.
